(19) United States
(12) Patent Application Publication
(10) Pub. No.: US 2003/0118594 A1
(43) Pub. Date: Jun. 26, 2003

(54) STABLE FORMULATIONS OF MHC-PEPTIDE COMPLEXES

(76) Inventors: BISHWAJIT NAG, FREMONT, CA (US); JEFFREY L. WINKELHAKE, REDWOOD CITY, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 08/485,605

(22) Filed: Jun. 7, 1995

(51) Int. Cl. A61K 39/00; A61K 31/7012

(52) U.S. Cl. 424/184.1; 514/53

(57) ABSTRACT

The present invention provides stable pharmaceutical compositions comprising MHC-peptide complexes and an alkylglycoside detergent. The compositions are useful for inhibiting T-cell mediated immune response associated with various disease, such as autoimmunity, and allergies.

### BACKGROUND OF THE INVENTION

The present invention relates to the preparation and use of pharmaceutical compositions comprising MHC-peptide complexes. In particular, the invention relates to new pharmaceutical compositions having increased solubility.

The major histocompatibility complex (MHC) class II antigens are heterodimeric cell surface glycoproteins involved in presenting antigenic peptides to CD4 positive T cells. The association of particular MHC alleles with a number of immunopathologies such as autoimmune disease, allergies, and the like is known. More than 30 autoimmune diseases are known, including, for example, rheumatoid arthritis (RA), myasthenia gravis (MG), systemic lupus erythematosus (SLE), insulin-dependent diabetes mellitus (IDDM) and multiple sclerosis (MS). Examples of allergic conditions include food hypersensitivities such as celiac disease and crohn disease and allergic responses to ragweed, dust mites, cats, honey bee venom, and grass pollen. The association between particular MHC alleles and various autoimmune diseases and allergies has been identified. In addition, the autoantigens and allergens for these diseases have been extensively studied.

Methods for identifying and inhibiting those aspects of the immune system responsible for undesirable T cell mediated immune responses, using MHC molecules complexed with antigenic peptides, have been described, See, e.g., U.S. Pat. Nos. 5,130,297, 5,194,425, and 5,260,422. The complexes comprise (1) an effective portion of the MHC-encoded glycoprotein; and (2) a peptide representing a fragment of antigen associated with the disease state to be treated (e.g., an autoantigenic peptide). These complexes have been shown to bind T cell receptors and cause non-responsiveness in target T-lymphocytes, thus ameliorating the disease. In addition, the complexes may contain an effector component which is generally a toxin or a label. The effector portion may be conjugated to either the MHC-encoded glycoprotein or to the antigenic peptide.

In many instances, the MHC component of the complex will comprise hydrophobic regions (e.g., the transmembrane region) that will cause the MHC-peptide complexes to aggregate in aqueous solutions. Solubilization of transmembrane containing membrane glycoproteins for drug development still remains a challenging problem to be solved for various clinical applications. Two commonly used iv. injectable detergents which are either approved by FDA or in trials are SDS and polyoxyethylenes (Tween-20 or Tween-80. SDS is a denaturing cationic detergent whereas Tween-20 or Tween-80 are non-ionic polyoxyethylenes containing laurate or oleate side chains. Improved methods for preparing pharmaceutical compositions comprising MHC-peptide complexes would be useful in improving their activity. The present invention addresses these and other needs.

### SUMMARY OF THE INVENTION

The present invention provides a pharmaceutical composition that includes a pharmaceutically acceptable carrier, a therapeutically effective amount an MHC-peptide complex (consisting of a preselected antigenic peptide and an isolated MHC Class II component) and an alkylglycoside detergent.

The alkylglycoside detergent can include an alkyl chain of between about 6 and about 14 carbon atoms and a disaccharide such as dodecyl β-D maltoside (DM). The pharmaceutically acceptable carrier is usually an aqueous carrier such as phosphate-buffered saline. The MHC-peptide complex consists of a preselected antigenic peptide that is typically between about 8 and 20 amino acids in length. The antigenic peptide can form a complex with an isolated MHC componant, especially an MHC Class II component such as HLA-DR2. An example of a MHC-peptide complex is DR2-MBP(83-102)Y⁸³. In a typical formulation of the pharmaceutical composition the MHC-peptide complex is present at a concentration between about 0.001% to about 5% by weight in a diluent consisting of 0.05% n-Dodecyl β-D-Maltoside, 0.008 M Sodium Phosphate-Dibasic 7-Hydrate, 0.002 M Sodium Phosphate-Monobasic-Monohydrate, 0.15 M Sodium Chloride in water, adjusted to pH 7.5 to 8.5.

In another aspect the invention provides a method of inhibiting a T cell-mediated immune response in a mammal by administering (e.g., intravenously) a pharmaceutical composition that includes a pharmaceutically acceptable carrier, a therapeutically effective amount an MHC-peptide complex (e.g., a preselected antigenic peptide and an isolated MHC Class II component) and an alkylglycoside detergent (e.g., DM). This method is useful for inhibiting a T cell-mediated immune response associated with an autoimmune disease, for example multiple sclerosis. For example, a pharmaceutical composition including DR2-MBP(83-102)Y⁸³ and dodecyl β-D maltoside (about 0.01% to 0.2% by weight) may be administered to a patient to diagnose or treat multiple sclerosis.

### BRIEF DESCRIPTION OF THE DRAWINGS

FIG. 1 shows the structures and physical properties of detergents tested for their ability to solubilize MHC-peptide complexes.

FIG. 2 shows the results of size exclusion HPLC analysis of MHC molecules (HLA-DR2) prepared with three non-ionic detergent (dodecyl β-D maltoside, octyl glucoside and Tween-80).

FIG. 3 shows the results of SDS-PAGE analysis of MHC molecules (HLA-DR2) prepared with three non-ionic detergent (dodecyl β-D maltoside, octyl glucoside and Tween-80).

FIG. 4 shows the results of size exclusion HPLC analysis of MHC molecules (HLA-DR2) prepared with various concentrations of dodecyl β-D maltoside.

FIG. 5 shows the results of SDS-PAGE analysis of MHC molecules (HLA-DR2) prepared with various concentrations of dodecyl β-D maltoside.

FIG. 6 shows the results of size exclusion HPLC analysis of MHC-peptide complexes, HLA-DR2 and MBP(83-102)Y⁸³ in formulations comprising dodecyl β-D maltoside and stored for up to 56 days.

FIG. 7 shows γIFN production in T cells contacted with MHC-peptide complexes in dodecyl β-D maltoside, octylglucoside, and Tween 80.

### DESCRIPTION OF THE PREFERRED EMBODIMENT

The present invention provides methods for preparing stable pharmaceutical compositions comprising MHC-peptide complexes. Pharmaceutical compositions of the invention can be used to modulate T cell function in the treatment of immunological disorders such as autoimmune diseases, allergic responses and transplant rejections.

Complexes and methods have been described that are useful for identifying and inhibiting those aspects of the immune system that are responsible for undesirable immune responses, such as autoimmunity. See, U.S. Pat. Nos. 5,130,297, 5,194,425, 5,284,935 and 5,260,422. These complexes and methods are designed to target T helper cells which recognize a particular antigen in association with a glycoprotein encoded by the MHC. The complexes effectively bind T cell receptors and cause non-responsiveness in target T-lymphocytes and other cells of the immune system.

The complexes used in the present invention contain at least two components: (1) a peptide representing a fragment of an autoantigen or other antigenic sequence associated with the disease state to be treated (i.e., an antigenic peptide); and (2) an effective portion of an MHC-encoded glycoprotein involved in antigen presentation. An effective portion of an MHC glycoprotein is one which comprises an antigen binding site and the regions necessary for recognition of the MHC-peptide complex by the appropriate T cell receptor. The MHC component can be either a Class I or a Class II molecule. The association between the peptide antigen and the antigen binding site of the MHC protein can be by covalent or noncovalent bonding. Additionally, the MHC-peptide complex may contain an effector component which is generally a toxin or a label. The effector portion may be conjugated to either the MHC-encoded glycoprotein or to the autoantigenic peptide. Complexes containing an effector component are disclosed and claimed in U.S. Pat. No. 5,194,425, supra.

Each component of the MHC-peptide complexes will be described in detail below.

The MHC-Derived Component:

Usually, the MHC component is isolated from a natural antigen presenting cell (e.g., a B cell, a dendritic cell, or a macrophage) or an immortalized cell line derived from such a cell. Alternatively the MHC component can be recombinantly produced. The term “isolated MHC component” as used herein refers to an MHC glycoprotein or an effective portion of an MHC glycoprotein (i.e., one comprising an antigen binding site or sites and the sequences necessary for recognition by the appropriate T cell receptor) which is in other than its native state (i.e., not associated with the cell membrane of the cell that normally expresses MHC). As described in detail below, the MHC component may be solubilized from an appropriate cell source or can be recombinantly produced. For human MHC molecules, human lymphoblastoid cells are particularly preferred as sources for the MHC component.

MHC glycoproteins have been isolated from a multiplicity of cells using a variety of techniques including, for example, solubilization by treatment with papain, by treatment with 3M KC1 and by treatment with nonionic detergents, such as, for example, NP-40, TWEEN® 80 and the like. The MHC molecules are then purified by affinity chromatography, using a column containing antibodies raised against the desired MHC molecule.

The present invention is based in part on the discovery that certain nonionic detergents, alkylglycosides, provide improved solubility and activity of isolated MHC-peptide complexes. Alkylglycosides as used herein refer to a class of amphipathic compounds that consist of an aliphatic hydrocarbon chain attached to a mono- or disaccharide. These compounds are mild, nonionic detergents and are metabolized to nontoxic alcohols and sugars.

The hydrophobicity of alkylglycosides increases as the alkyl chain length increases. Alkylglycosides used in the compositions of the invention will contain aliphatic chains, saturated and unsaturated, consisting of between about 4 and about 24 carbon atoms, typically between about 6 and about 15, usually between about 8 and about 12. The carbohydrate moiety can consist of any number of monosaccharides, such as glucose, galactose, fucose, fructose, and the like. Disaccharides include, sucrose, maltose, and the like. Usually, compounds comprising a disaccharide will be more soluble than those comprising a monosaccharide. The sugars in the carbohydrate moiety can be variously substituted, as well. For instance, the carbohydrate moiety may comprise amino sugars, such as glucosamine, galactosamine and the like.

A number of alkylglycosides are commercially available including, but not limited to, tetradecylmaltoside, tridecylmaltoside, dodecylmaltoside, decylmaltoside, octylmaltoside, dodecylsucrose, decylsucrose, heptylglucoside, nonylglucoside, and hexylglucoside. Other suitable commercially available compounds include, for example, deconyl-N-methylglucamide, heptanoyl-N-methylglucamide, nonanoyl-N-methylglucamide, and heptylthioglucoside. A preferred alkylglycoside is dodecyl β-D maltoside (DM). As shown below, MHC-peptide complexes prepared in composition comprising DM have increased solubility and activity.

Althought alkylglycosides are readily availabel from commercial sources, they can also be synthesized according to methods which are known to those of skill in the art. Compounds such as DM, having a hydrocarbon chain attached to an anomeric center of a sugar can be prepared by reacting the parent sugar with a hydrocarbon alcohol under mild acidic conditions to provide the desired products having an attached hydrocarbon chain. Alternatively, the hydroxyl functionality at the anomeric carbon can be alkylated with, for example, an alkyl halide such as dodecyl iodide. These alkylations will, in some instances, require the use of protecting groups on the remaining hydroxyl functionalities to prevent competing reaction. Following alkylation of the anomeric hydroxyl group, the protecting groups can be removed. The protecting groups used in the present invention are know to those of skill in the art and can be any of those groups described in Greene, et al., Protective Groups In Organic Chemistry, 2nd Ed., John Wiley & Sons, New York, N.Y., 1991.

In addition to carbohydrates having the aliphatic chain attached to the anomeric hydroxyl group, the chain can be attached to a hydroxyl group at any of the remaining carbon atoms. Typically, the selective alkylation of a carbohydrate hydroxyl group will involve protection of the remaining hydroxyl centers. This may also include initial protection of the desired reactive center, followed by subsequent protection of the remaining centers and selective removal of the protecting group attached to the reactive center of interest.

One of skill in the art will also understand that the hydrocarbon chains used to prepare the alkylglycosides include both alkyl groups and the corresponding acyl groups which are derived from C₈ to C₂₄ carboxylic acids. Acylation of selected sites on the sugar moieties with a long chain acyl group can be carried out using protection/deprotection strategies similar to those described above. The reactive acyl group is typically an acyl halide or other activated fatty acid ester (for example, palmitoic acid p-nitrophenyl ester). Additionally, the alkyl and acyl groups may have one or more sites of unsaturation. Examples of suitable groups include palmitoyl, stearoyl, oleoyl, myristoyl, caproyl, decyl, dodecyl, heptyl, octyl, nonyl, undecyl and tetradecyl.

The particular MHC molecule used in the complexes of the invention will depend upon the disease to be treated. A number of autoimmune diseases are correlated with specific MHC types. Methods for identifying which alleles, and subsequently which MHC encoded polypeptides, are associated with an autoimmune disease are known in the art. For example, susceptibility to insulin-dependent diabetes mellitus is linked to DR3 and DR4 haplotypes. The haplotype DR2W2 is associated with myasthenia gravis. Several class II serotypes have been associated with rheumatoid arthritis, including DR4(Dw4), DR4(Dw14) and DR1. DR2 and DR3 individuals are at a higher risk than the general population to develop systemic lupus erythematosus. The DR2 haplotype is associated with multiple sclerosis.

The Antigenic Peptide:

It is believed that the presentation of antigen by the MHC glycoprotein on the surface of antigen-presenting cells (APCs) occurs subsequent to the hydrolysis of antigenic proteins into smaller peptide units. The location of these smaller segments within the antigenic protein can be determined empirically. These segments are thought to be about 8 to about 18 residues in length and to contain both the agretope (recognized by the MHC molecule) and the epitope (recognized by the T cell receptor on the T-helper cell). The length of peptides capable of binding an MHC molecule, however, can vary. Thus, peptides of greater length, e.g., up to 100 residues can also be used in the complexes. Usually, the peptides will be less than about 50 residues in length, preferably less than about 30, most preferably between about 8 and 20 residues in length.

Antigenic proteins or tissues for a number of autoimmune diseases and allergies are known. Using standard procedures one of skill in the art can readily determine the relevant antigenic peptide for the antigens associated with many immune disorders. For instance, in multiple sclerosis, which results in the destruction of the myelin sheath in the central nervous system, it is known that myelin basic protein (MBP) (i.e., the major protein component of myelin) and proteolipid protein (PLP) are the principal autoantigens. Autoantigenic peptides for treatment of MS comprise amino aids 84-102 and 148-162 of MBP. The examples below, describe preferred complexes for the treatment of MS comprising DR2 and MBP 83-102, in which residue 83 is substituted with tyrosine. This antigenic peptide is designated MBP(83-102)Y⁸³. The formula of MBP(83-102)Y⁸³ is Try-Asp-Pro-Val-Val-His-Phe-Phe-Lys-Asn-Ile-Val-Thr-Pro-Arg-Thr-Pro-Pro-Pro-Ser. In a preferred embodiment the tyrosine is N-acetyl-tyrosine.

Systemic lupus erythematosus has a complex symptomology, it is known to result from an autoimmune response to red blood cells. Peptides which are the antigenic effectors of this disease are found in the proteins on the surface of red blood cells. Rheumatoid arthritis (RA), a chronic inflammatory disease, results from an immune response to proteins found in the synovial fluid. Insulin-dependent diabetes mellitus (IDDM) results from autoimmune attack on the beta cells within the Islets of Langerhans which are responsible for the secretion of insulin. Circulating antibodies to Islets cells surface antigens and to insulin are known to precede IDDM. Critical peptides in eliciting the immune response in IDDM are believed to be portions of the insulin sequence and the beta cell membrane surface proteins.

Once determined, the relevant antigenic peptide subunits can be readily synthesized using standard automated methods for peptide synthesis being that they are relatively short in length. Alternatively, they can be made recombinantly using isolated or synthetic DNA sequences, but this is not the most efficient approach for peptides of this length.

The Effector Component:

Additionally, the complexes of the invention can be designed to destroy the immune response to the peptide in question. In this instance, the MHC-peptide complex will contain an effector component. The effector portion of the MHC-peptide molecule can be, for example, a toxin, a chemotherapeutic agent, an antibody to a cytotoxic T-cell surface molecule, a lipase, or a radioisotope emitting “hard” radiation (e.g., beta radiation). A number of protein toxins are well known in the art and include, for example, ricin, diphtheria, gelonin, Pseudomonas toxin, and abrin. Chemotherapeutic agents include, but are not limited to, doxorubicin, daunorubicin, methotrexate, cytotoxin, and anti-sense RNA. Moreover, antibiotics can also be used as the effector component. Antibodies have been isolated to cytotoxic T-cell surface molecules and these may thus operate as toxins. In addition, radioisotopes such as yttrium-90, phosphorus-32, lead-212, iodine-131, or palladium-109 can be used. The emitted radiation effects the destruction of the target T-cells.

In some cases the active portion of the effector component is entrapped in a delivery system such as a liposome or dextran carrier; in these cases, either the active component or the carrier may be bound in the complex.

If the effector molecule is intended to be a label, a gamma-emitting radioisotope such as technetium-99 or indium-111 can be used. In addition, other types of labeling such as fluorescence labeling by using, for example, fluorescein.

The effector component can be attached to the MHC glycoprotein or, if its nature is suitable, to the peptide portion. Iodine 131 or other radioactive labels, for example, can often be included in the peptide determinant sequence. Complexes containing an effector component are disclosed and claimed in U.S. Pat. No. 5,194,425, supra.

Formation of the MHC-Peptide Complex:

Once the MHC component has been isolated and the antigenic peptide has been synthesized, these two elements can be associated with one another to form an MHC-peptide complex by means known in the art, as described in U.S. Pat. No. 5,130,297, supra. The antigenic peptides can be associated noncovalently with the pocket portion of the MHC protein by, for example, mixing the two components. Excess peptide can be removed by any of a number of standard procedures, such as ultrafiltration or dialysis. The peptides can also be covalently bound using standard procedures by, for example, photo affinity labelling, (see, e.g., Hall et al., Biochemistry 24:5702-5711 (1985).

Formulation and Administration of the MHC-Peptide Complex:

Administration of the complexes of the invention is usually systemic and may be effected by standard methods known to those of skill in the art. Standard formulations for various modes of administration are found in Remington's Pharmaceutical Sciences, (Mack Publishing Company, Philadelphia, Pa., 17th ed. (1985)). A variety of pharmaceutical compositions comprising complexes of the present invention and pharmaceutically effective carriers can be prepared.

In preparing pharmaceutical compositions comprising MHC-peptide complexes, it is frequently desirable to modify the complexes to alter their pharmacokinetics and biodistribution. For a general discussion of pharmacokinetics, see, Remington's Pharmaceutical Sciences, supra, Chapters 37-39. A number of methods for altering pharmacokinetics and biodistribution are known to one of ordinary skill in the art.

The pharmaceutical compositions are intended for parenteral, topical, oral or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, and capsules.

Preferably, the pharmaceutical compositions are administered intravenously. Thus, compositions for intravenous administration are provided which comprise a solution of the MHC-peptide complex dissolved or suspended in an acceptable carrier, preferably an aqueous carrier. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, and the like. A variety of aqueous carriers may be used, e.g., water, buffered water, 0.4% saline, PBS, and the like.

These compositions may be sterilized by conventional, well-known sterilization techniques, usually filter sterilization. The resulting aqueous solutions may be packaged for use as is, or may be lyophilized. The lyophilized preparations can be combined with a sterile aqueous solution prior to administration.

The concentration of the complex can vary widely, i.e., from less than about 0.05%, usually at or at least about 1% to as much as 10 to 30% by weight and will be selected primarily by fluid volumes, viscosities, etc., in accordance with the particular mode of administration selected. Preferred concentrations for intravenous administration are about 0.02% to about 0.1% or more in a suitable aqueous carrier.

Preferred formulations comprise an aqueous carrier and a nonionic detergent, preferably an alkylglycoside. The alkylglycoside is preferably present at a concentration above its critical micellar concentration (cmc). A preferred alkylglycoside is DM (available from SAF (Sigma/Aldrich/Fluga) Bulk Chemicals, 3050 Spreuce Street, St. Louis, Mo. 63103). Typically, the DM will be present at a concentration between about 0.01% to about 0.1% by weight, preferably between about 0.02% and about 0.05%.

A particularly preferred formulation comprises MHC-peptide complexes in the following diluent:

| | | | |
|---|---|---|---|
| n-Dodecyl β-D-Maltoside | 0.05% | 0.5 | mg |
| Sodium Phosphate-Dibasic 7-Hydrate | 0.008 M | 2.27 | mg |
| Sodium Phosphate-Monobasic-Monohydrate | 0.002 M | 0.25 | mg |
| Sodium Chloride | 0.15 M | 8.7 | mg |
| Water for injection | | 1.0 | ml |

The diluent is adjusted to a pH between about 7.5 and about 8.5 and is a clear and colorless liquid.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient. For aerosol administration, the complexes are preferably supplied in finely divided form along with a surfactant and propellant.

The compositions containing the complexes can be administered for therapeutic, prophylactic, or diagnostic applications. In therapeutic applications, compositions are administered to a patient already suffering from a disease, as described above, in an amount sufficient to cure or at least partially arrest the symptoms of the disease and its complications. An amount adequate to accomplish this is defined as “therapeutically effective dose.” Amounts effective for this use will depend on the severity of the disease and the weight and general state of the patient. This will typically be between about 0.1 μg/kg and about 10 mg/kg, preferably about 0.2 to about 500 μg/kg. For example, for administering HLA-DR2/MBP(83-102)Y83 complexes the dose will typically be between about 0.2 μg/kg to about 20 μg/kg. For a patient of average weight the dose will typically be between about 16 and 2000 μg. A particularly preferred schedule for administering the composition is by a slow intravenous bolus (about 0.1 ml/sec for about 10 seconds) administered every other day.

In prophylactic applications, compositions containing the complexes of the invention are administered to a patient susceptible to or otherwise at risk of a particular disease. Such an amount is defined to be a “prophylactically effective dose.” In this use, the precise amounts again depend on the patient's state of health and weight. The doses will generally be in the ranges set forth above.

In diagnostic applications, compositions containing the appropriately complexes or a cocktail thereof are administered to a patient suspected of having an autoimmune disease state to determine the presence of autoreactive T cells associated with the disease. Alternatively, the efficacy of a particular treatment can be monitored. An amount sufficient to accomplish this is defined to be a “diagnostically effective dose.” In this use, the precise amounts will depend upon the patient's state of health and the like, but generally range from 0.01 to 1000 mg per dose, especially about 10 to about 100 mg per dose.

This invention will be described in greater detail by way of specific examples. The following examples are offered for illustrative purposes, and is intended neither to limit or define the invention in any manner.

### EXAMPLES

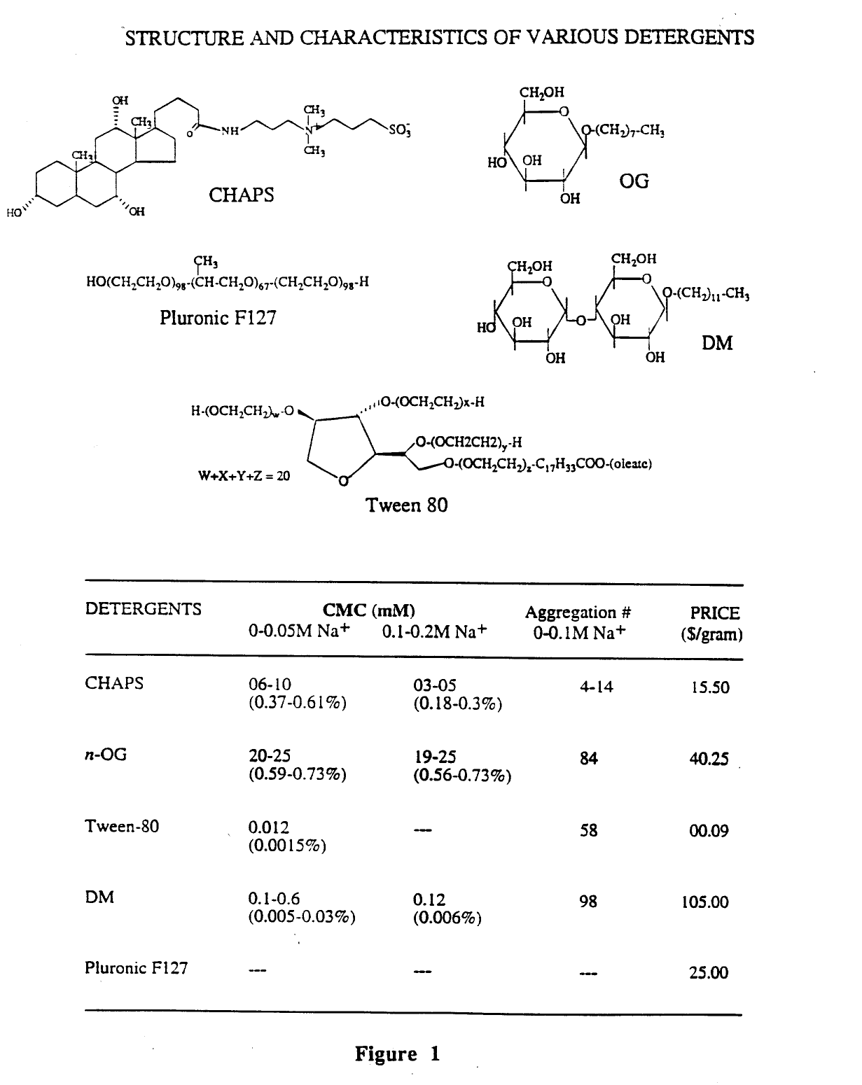

Several preclinical studies have demonstrated that complexes of affinity purified MHC class II and antigenic peptide can be used for prevention and treatment of various autoimmune diseases. A number of detergents were studied for their ability to solubilize human HLA-DR2 and MHC-peptide complexes. In addition the complexes prepared in the detergents were assayed for activity. A summary of structural and physical characteristics of various detergents used for solubilization of HLA-DR2 is presented in FIG. 1. Pluronic F-127 and CHAPS detergents were found to be toxic to T-cells and were not studied further.

Aggregation Assays

Three non-ionic detergents (DM, OG, and Tween-80) were tested. The experiments were carried out as generally described in U.S. Pat. Nos. 5,130,297, 5,194,425, 5,260,422 and 5,284,935, all of which are incorporated herein by reference. Briefly, HLA-DR2 was purified from EBV-transformed lymphoblastoid cells [GM03107] obtained from the National Institute of General Medical Sciences human genetic mutant cell repository (Corinell Institute of Medical Research, NJ.). A triton X-100 cell lysate was prepared and applied to a L243 coupled sepharose-4B column and the bound DR2 was eluted in phosphate buffer containing 0.05% n-dodecyl β-D-maltoside (DM) detergent at pH 11.3. Fractions were immediately neutralized with 20% monobasic phosphate and the DR2 pool was collected through a DEAE ion exchange column in 20 mM phosphate buffer containing 0.5 M NaCl and 0.05% DM, pH 6.0. The flow through purified protein solution from the DEAE column was neutralized with 10% tribasic sodium phosphate, filtered through a 180 kD membrane and concentrated to 1 mg/ml. The final DR2 preparation was characterized by 13.5% SDS polyacrylamide gel electrophoresis followed by silver staining.

To prepare the DR2-peptide complex, purified DR2 was dialyzed against 100 mM citrate buffer pH 6.0 containing 0.05% DM for 24 hours at 4° C. prior to the addition of MBP(83-102)Y⁸³ peptide. The reaction mixture was incubated at 37° C. for 96 hours and free peptide was removed by Sephacryl S200 gel filtration.

Results

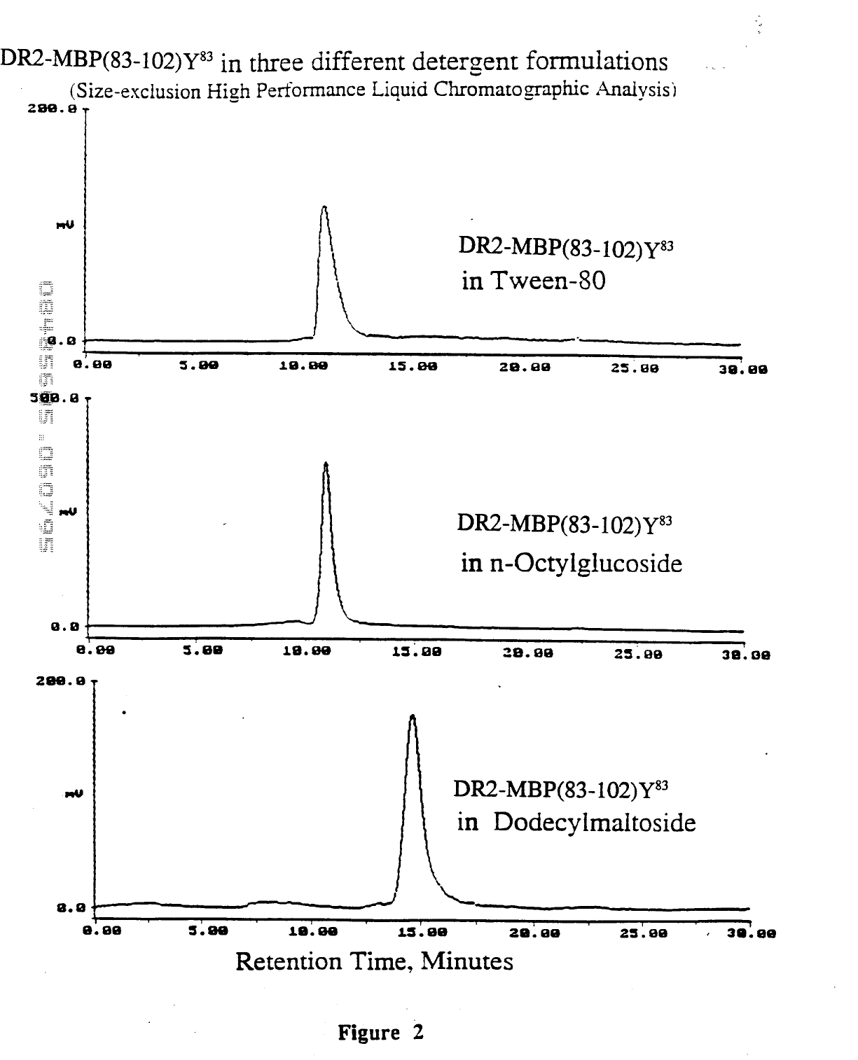

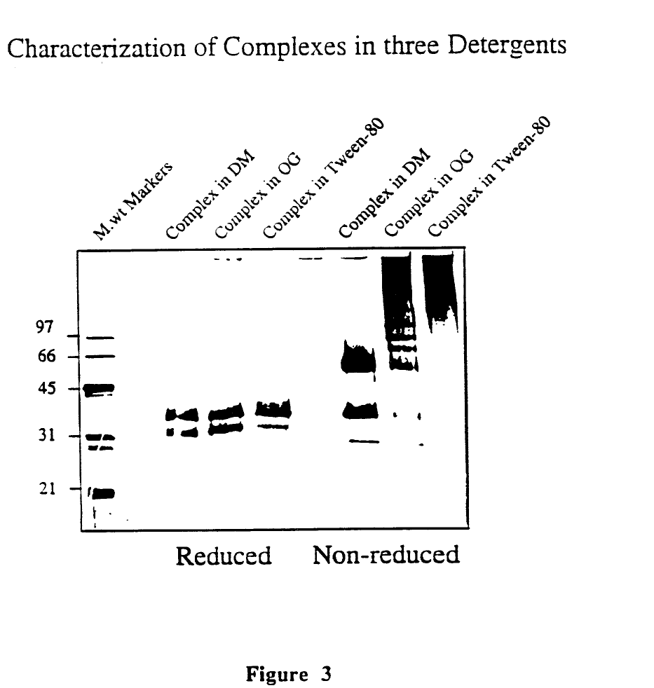

A formulation containing 0.05% DM resulted in minimal level of aggregated complexes of HLA-DR2 and MBP(83-102)Y⁸³ peptide as measured by size-exclusion HPLC analysis (FIG. 2). Similar results were observed when complexes of MHC II-peptide prepared in the three detergents were tested in SDS-PAGE under non-reduced conditions. Based on these results, DM detergent was selected for further studies (FIG. 3).

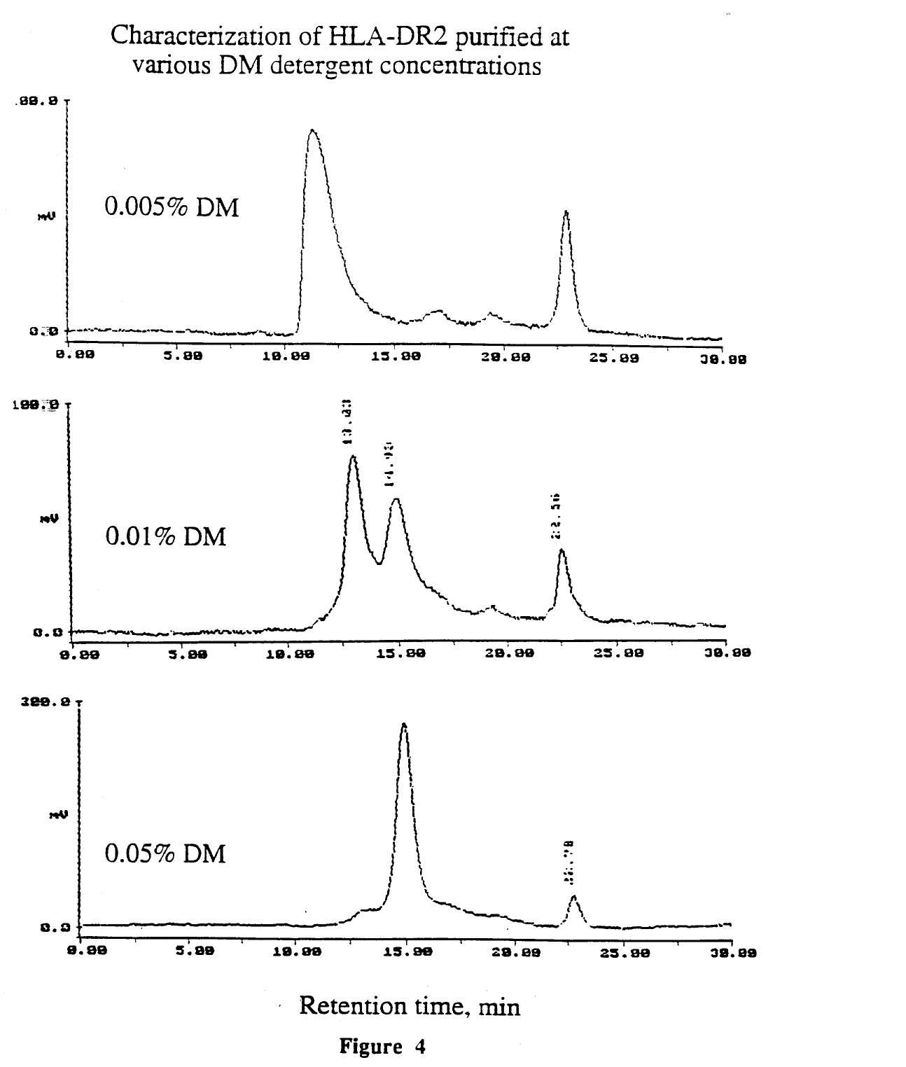

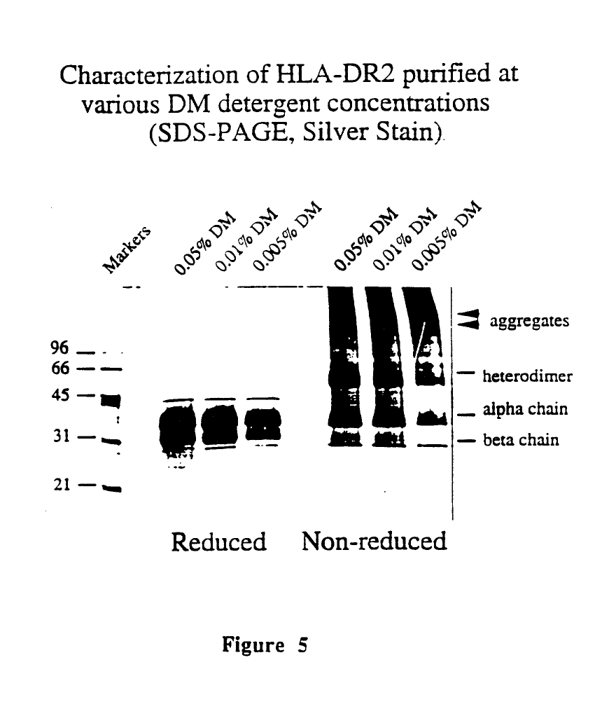

HLA-DR2 was purified using the methods described above in increasing concentrations of DM detergent to determine the minimum concentration required for complete solubilization with respect to the aggregation level. As shown by HPLC in FIG. 4, the level of DR2 aggregation is dependent on DM concentration. The HPLC results correlate well with the SDS-PAGE analysis (FIG. 5). Lowering the DM concentration below 0.05% resulted in increased aggregation level of DR2. Based on these results, a DM concentration of 0.05% was selected for the final formulation.

Stability of MHC-peptide Complexes in DM

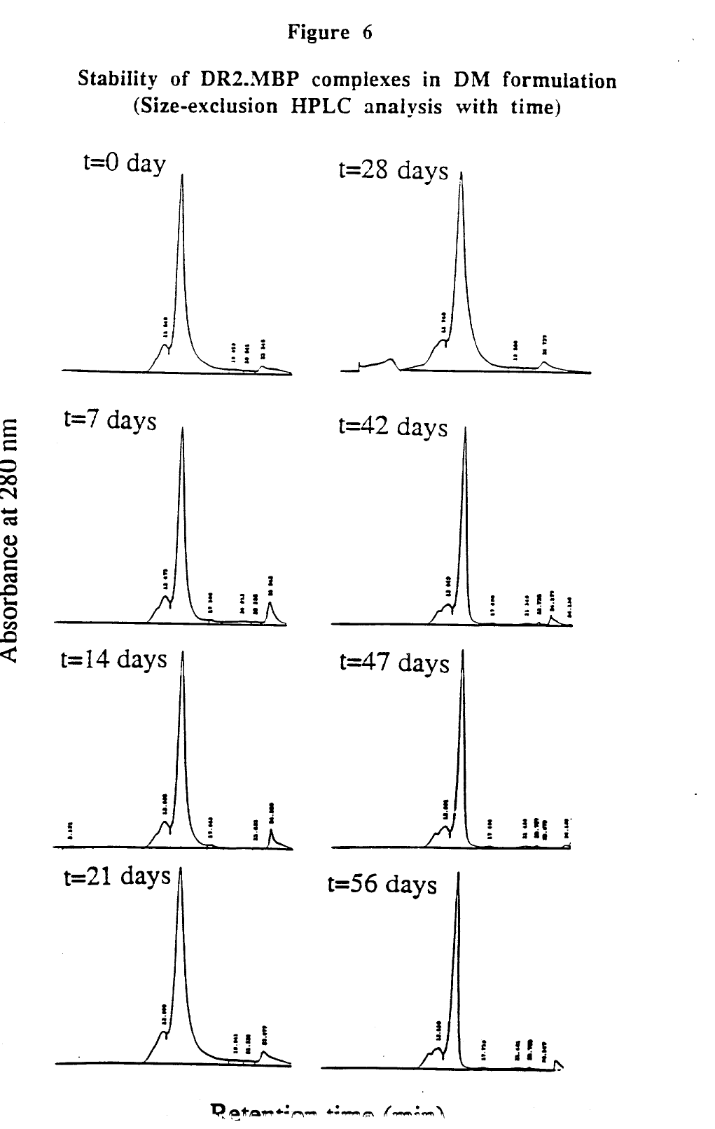

The stability of purified complexes of HLA-DR2 and MBP(83-102)Y⁸³ prepared in 0.05% DM formulation with respect to the aggregation level was analyzed by size exclusion HPLC. Results presented in FIG. 6 show that complexes stored at 4° C. for 8 weeks did not tend to aggregate following purification.

Functional Assays

In order to test the functional activity of complexes prepared in various detergents, a T cell receptor (TCR) occupancy assay was performed using H. saimiri virus (HSV) transformed cloned human T cells (SS8T) which are restricted for HLA-DR2 and MBP(84-102) peptide in a dose dependent manner. Complexes of DR2 and MBP(83-102)Y⁸³ peptide were prepared in DM, OG, and Tween-80 detergents, and were assayed for the increase in gamma-IFN level as a measure of the TCR occupancy. An increase in gamma IFN production has been shown to occur following TCR occupancy by specific ligands. In particular, complexes of native DR2 with MBP(83-102)Y⁸³ peptide have been shown to lead to increase IFN production.

The T cells were cultured in RPMI 1640 medium supplemented with 2 mM L-glutamine, 100 units/ml penicillin, 100 μg/ml streptomycin, 10% fetal bovine serum (Hyclone) and 50 units/ml human IL-2 (ABI) at 37° C. Every alternate day the cells were transferred to fresh media. The complex preparations were added at a final concentration of 10% v/v in a microtiter tissue culture plate and the cells were added at a density of 20,000/well in 200 μl media without IL-2. After 48 hours incubation at 37° C., the supernatants were collected from each well to test for the increase in gamma-IFN level.

For the detection of gamma-IFN, Nunc Maxisorb 96 well plates were coated with anti-human gamma-IFN monoclonal antibody at a concentration of 0.5 μg/well and incubated at 4° C. overnight. The wells were blocked with 0.1% BSA, and samples were incubated at room temperature for 2 hours. The standard curve was generated by using human gamma-IFN with a dilution range of 1000 , 500, 100, 50, 10, 5, 1, 0.5, 0.1 units/ml (270 units/ml=10.75 ng/ml ). Rabbit anti-human gamma-IFN was then added at a concentration of 1 μg/ml and plates were incubated at room temperature for additional 2 hours. Wells were extensively washed and incubated with HRP-conjugated goat anti-rabbit at a concentration of 800 ng/ml for 1 hour at 37° C., prior to the color developed using TMB as a substrate. The reaction was stopped by 2 N sulfuric acid at 5 min. and the absorbance was measured at 450 nm.

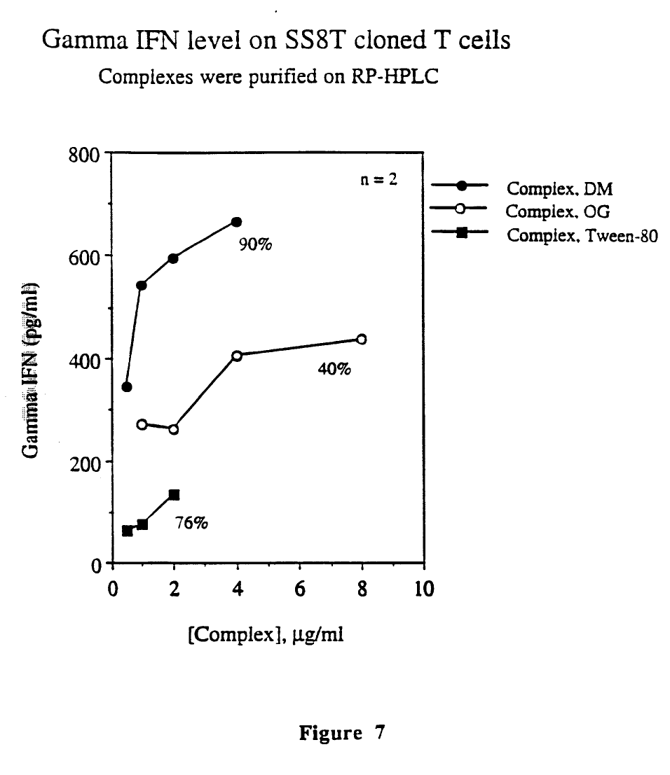

As shown in FIG. 7, complexes prepared in DM formulation showed significant increased gamma-IFN level when exposed to SS8T cloned T cells. As noted in the figure, the occupancy of the MHC molecules (i.e., proportion of the molecules complexed with peptide) was 90% in DM, 40% in octylglucoside and 76% in Tween-80). This experiment demonstrates that in addition to reducing aggregation of MHC molecules DM increases the loading of the MHC molecules (i.e., formation of MHC/peptide complexes) by enhancing binding of the peptide.

Safety and Toxicity Studies

Safety and toxicity studies were carried out for the intravenous administration of dodecyl-β-D-maltoside in various animal models. Results summarized in Table 1 show that the i.v. administration of DM detergent at various tested doses had no toxic effects.

Results presented here demonstrate that the formulation containing 0.05% dodecyl β-D-maltoside provide stable homogenous complexes of MHC-peptide. The formulation in 0.05% DM has no adverse toxic effect and can be utilized in intravenous administration of MHC-peptide complexes for various clinical studies.

TABLE 1
SAFETY PROFILE - Intravenous Dodecyl-β-D-Maltoside

| | | | | | | |
|---|---|---|---|---|---|---|
| | | | Dose | | | |
| Test | Species | N | (ug/kg)* | Schedule | Status | Results |

1.) GLP Toxicology

| | | | | | | |
|---|---|---|---|---|---|---|
| (a). Acute Range-finding | mouse | 10/sex | 400 | bolus | Done | Draft Final Aud |
| | | | | | | poss kindey cortical |
| | | | | | | tubules |
| (b). Subacute - full histo | mouse | 10 | 4 | QD14 | in histopath | No Eff @ necr/clin-path |
| | | 10 | 40 | QD14 | ″ | No Effects |
| | | 10 | 400 | QD14 | ″ | D14 Spleen wt slightly |
| | | | | | | lower than controls |
| | | | | | | returned to normal by |
| | | | | | | week 4/Draft Rept 12/16 |

| | | |
|---|---|---|
| (c). Subacute | monkey | planned with testing of formulated product |

2.) Safety Pharmacology (Acute Effects) Screen:**

| | | | | | | |
|---|---|---|---|---|---|---|
| (a). Neuropharm profile | mouse | 10 | 10 | bolus | Done | No Effects |
| | | | 100 | | | No Effects |
| (b). Diuretic Assay | rat | 10 | 10 | bolus | 10/24 | No Effects |
| | | | 100 | | | No Effects |
| (c). Barbit Sleep Potent | mouse | 10 | 10 | bolus | Done | No Effects |
| | | | 100 | | | No Effects |
| (e). Cardiovasc Effects | dog | 3 | 10 | bolus | 10/25 | No Effects |

| | |
|---|---|
| up if NOEL to 100 | No Effects |

*Clinical dose = 7 μg/kg Q2Dx3 = 21 μg/week
**same profiles planned for product - extended N's

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

What is claimed is:

1. A pharmaceutical composition comprising a pharmeceutically acceptable carrier, a therapeutically effective amount an MHC-peptide complex and an alkylglycoside detergent, the MHC-peptid complex consisting of a preselected antigenic peptide and an isolated MHC Class II component.

2. The pharmaceutical composition of claim 1, wherein the antigenic peptide consists of between about 8 and about 20 amino acids.

3. The pharmaceutical composition of claim 1, wherein the MHC component is DR2.

4. The pharmaceutical composition of claim 1, wherein the MHC-peptide complex is DR2-MBP(83-102)Y⁸³.

5. The pharmaceutical composition of claim 1, wherein the alkylglycoside detergent comprises an alkyl chain of between about 6 and about 14 carbon atoms.

6. The pharmaceutical composition of claim 1, wherein the alkylglycoside detergent comprises a disaccharide.

7. The pharmaceutical composition of claim 1, wherein the alkylglycoside is dodecyl β-D maltoside.

8. The pharmaceutical composition of claim 7, wherein the dodecyl β-D maltoside is present at a concentration between about 0.01% and about 0.1% by weight.

9. The pharmaceutical composition of claim 1, wherein the MHC-peptide complex is present at a concentration between about 0.001% to about 0.2% weight.

10. The pharmaceutical composition of claim 1, wherein the aqueous carrier is phosphate-buffered saline.

11. The pharmaceutical composition of claim 1, which consists essentially of 0.05% n-Dodecyl β-D-Maltoside, 0.008 M Sodium Phosphate-Dibasic 7-Hydrate, 0.002 M Sodium Phosphate-Monobasic-Monohydrate, 0.15 M Sodium Chloride and water and between about 16 and 2000 μg/ml DR2-MBP(83-102)Y⁸³ complexes.

12. A method of inhibiting a T cell-mediated immune response in a mammal, the method comprising administering to the mammal a pharmaceutical composition comprising a pharmaceutically acceptable carrier, a therapeutically effective amount an MHC-peptide complex and an alkylglycoside detergent, the MHC-peptide complex consisting of a preselected antigenic peptide and an isolated MHC Class II component.

13. The method of claim 12, wherein the composition is administered intravenously.

14. The method of claim 12, wherein the T cell-mediated immune response is associated with an autoimmune disease.

15. The method of claim 14, wherein the autoimmune disease is multiple sclerosis.

16. The method of claim 12, wherein the MHC-peptide complex is DR2-MBP(83-102)Y⁸³.

17. The method of claim 12, wherein the alkylglycoside is dodecyl β-D maltoside.

18. The method of claim 12, wherein the dodecyl β-D maltoside is present at a concentration between about 0.01% and about 0.1% by weight.

19. The method of claim 12, wherein the therapeutically effective amount is between about 0.015 μg MHC-peptide complex per kg body weight and about 20 μg MHC-peptide complex per kg body weight.


SEQUENCE LISTING (1 sequence)

SEQ ID NO: 1 - protein, 20 aa
  Modified-site 1: /product= “OTHER” /note= “Xaa = Tyr or N-acetyl-tyrosine”
  hypothetical: NO
XDPVVHFFKNIVTPRTPPPS